(19) United States
(12) Patent Application Publication
(10) Pub. No.: US 2019/0224454 A1
(43) Pub. Date: Jul. 25, 2019

(54) TUBE SECURING DEVICE AND METHOD OF USE

(71) Applicant: Research Institute at Nationwide Children's Hospital, Columbus, OH (US)

(72) Inventors: Robert V. Strouse, Columbus, OH (US); Jeremy Patterson, Columbus, OH (US); Ish K. Gulati, Hilliard, OH (US)

(21) Appl. No.: 16/335,797

(22) PCT Filed: Sep. 22, 2017
(86) PCT No.: PCT/US2017/052957

§ 371 Date: Mar. 22, 2019

Related U.S. Application Data
(60) Provisional application No. 62/398,145, filed on Sep. 22, 2016.

(51) Int. Cl. A61M 25/02

(52) U.S. Cl. CPC A61M 25/02; A61M 2025/0253; A61M 2025/024

(57) ABSTRACT

A tube securing device is disclosed. The device includes a tube receiving channel having a lower portion and an upper portion. The upper portion includes a plurality of flexible clasps that are adapted to retain tubing in the tube receiving channel. A footprint portion is secured to the tube receiving channel. The footprint portion is provided with non-woven fabric or an adhesive pad. A method of using the tube securing device is also disclosed.

### CROSS-REFERENCE TO RELATED APPLICATIONS

The present application claims priority to U.S. Provisional patent application Ser. No. 62/398,145 filed Sep. 22, 2016 entitled TUBE SECURING DEVICE AND METHOD OF USE. The entire contents of the above-identified application are incorporated herein by reference in its entirety for all purposes.

### FIELD OF THIS DISCLOSURE

The present disclosure relates to a securing system. More specifically, the present disclosure relates to a securing device having at least one tube receiving channel secured to a footprint portion that is adapted to be attached to a patient's skin. The securing device can be used to anchor tubing to a desired location on a patient's body.

### BACKGROUND

Various medical procedures sometimes necessitate the use of medical tubing (e.g., chest tubes, femoral lines, arterial lines, port lines and the like) on infants or patients with extremely weak skin tissue, such as the elderly or burn victims. Regardless of the age of the patient, it is always desirable to ensure that the position and/or location of the medical tubing is not inadvertently disturbed. It is known to use tape or sutures to secure the medical tubing in a desired position and/or location. However, the use of tape and/or sutures in this specific application is not ideal when working with weak tissue patients. For example, neonatal infants cannot utilize tape or sutures due to the fragility of their skin. Therefore, there is a need to provide an apparatus that can reliably secure medical tubing in a desired location while not damaging the fragile skin of an infant or weak skin tissue patient.

### SUMMARY

One aspect of the present disclosure is a device for securing medical tubing to a patient. The device includes a tube receiving channel that extends along a longitudinal axis between a first end and a second end. The tube receiving channel includes a lower portion and an upper portion. The upper portion has a plurality of flexible clasps that are spaced from one another along the longitudinal axis. A footprint portion is secured to the tube receiving channel. The footprint portion has a substantially hour-glass shaped perimeter. A plurality of reinforcement arms are provided on the footprint portion. Each of the plurality of reinforcement arms are curved so as to point toward one of the first end and the second end. A plurality of relief grooves are provided on the footprint portion. A single groove of the plurality of relief grooves is provided between adjacent reinforcement arras of the plurality of reinforcement arms. At least one of a non-woven fabric and an adhesive pad is secured to the footprint portion.

Another aspect of the present disclosure is another embodiment of a

device for securing medical tubing to a patient. The device includes a tube receiving channel that extends along a longitudinal axis between a first end and a second end. The tube receiving channel includes a lower portion and an upper portion. The upper portion has a plurality of flexible clasps that are spaced apart from one another along the longitudinal axis. A footprint portion is secured to the tube receiving channel. The footprint portion includes a plurality of arms that extend radially outward from a midpoint between the first end and the second end along the longitudinal axis. The footprint portion further includes a plurality of reinforcement hoops. The reinforcement hoops are concentric about the midpoint and interconnect adjacent arms of the plurality of arms. At least one of a non-woven fabric and an adhesive pad is secured to the footprint portion.

Yet another aspect of the present disclosure is a method of securing medical tubing to a patient. The method includes the step of providing a device having a tube receiving channel that includes a lower portion and an upper portion. The upper portion has a plurality of flexible clasps that are spaced apart from one another along a longitudinal axis. A footprint portion is secured to the tube receiving channel. A non-woven fabric is secured to the footprint portion. The method further includes the step of applying liquid skin protectant to a desired area of the patient's skin and allowing the liquid protectant to dry to form a solid protectant. The securing device is placed on the patient such that the non-woven fabric engages the solid protectant. Additional liquid protectant is applied to the interface between the non-woven fabric and the solid protectant so as to impregnate the non-woven fabric with additional liquid protectant, and the additional liquid protectant is allowed to dry. Medical tubing is inserted into the tube receiving channel.

### BRIEF DESCRIPTION OF THE SEVERAL VIEWS OF THE DRAWINGS

The foregoing and other features and advantages of the present disclosure will become apparent to one skilled in the art to which the present disclosure relates upon consideration of the following description of the disclosure with reference to the accompanying drawings, wherein like reference numerals, unless otherwise described refer to like parts throughout the drawings and in which:

FIG. 1 is a perspective view of a tube securing device in accordance with a first example embodiment of the present disclosure;

FIG. 2 is a top plan view of the tube securing device of FIG. 1;

FIG. 3 is a front elevation view of the tube securing device of FIG. 1;

FIG. 4 is a side elevation view of the tube securing device of FIG. 1;

FIG. 5A is a front perspective view of a first variation of the chest tribe securing device of FIG. 1;

FIG. 5B is a front perspective view of a second variation of the chest tube securing device of FIG. 1;

FIG. 6 is a front perspective view of a third variation of the chest tube securing device of FIG. 1;

FIG. 7 is a perspective view of a tube securing device in accordance with a second example embodiment of the present disclosure;

FIG. 8 is a perspective view of a tube securing device in accordance with a variation of the tube securing device of FIG. 7;

FIG. 9 is a perspective view of a tube securing device in accordance with a third example embodiment of the present disclosure;

FIG. 10 is a perspective view of a tube securing device in accordance with a variation of the tube securing device of FIG. 9;

FIG. 11 is a perspective view of a tube securing device in accordance with a fourth example embodiment of the present disclosure;

FIG. 12 is a perspective view of a tube receiving channel of a tube securing device in an open position in accordance with a fifth example embodiment of the present disclosure;

FIG. 13 is a perspective view of a tube receiving channel of a tube securing device in a closed position in accordance with the fifth example embodiment of the present disclosure;

FIG. 14 is a perspective view of a tube securing device in an open position in accordance with the fifth example embodiment of the present disclosure;

FIG. 15 is a perspective view of a tube securing device in a closed position in accordance with the fifth example embodiment of the present disclosure;

FIG. 16 is a perspective view of a tube securing device in an open position receiving a tube in accordance with the fifth example embodiment of the present disclosure;

FIG. 17 is a perspective view of a tube securing device in an open position housing a tube in accordance with the fifth example embodiment of the present disclosure;

FIG. 18 is a perspective view of a tube securing device in a closed position housing a tube in accordance with the fifth example embodiment of the present disclosure;

FIG. 19 is a perspective view of a tube securing device and a tube receiving channel, the tube securing device in an open position receiving a tube in accordance with a fifth example embodiment of the present disclosure;

FIG. 20 is a perspective view of a tube securing device and a tube receiving channel, the tube securing device in an open position housing a tube in accordance with a fifth example embodiment of the present disclosure;

FIG. 21 is a perspective view of a tube securing device and a tube receiving channel, the tube securing device in a closed position housing a tube in accordance with a fifth example embodiment of the present disclosure;

FIG. 22 illustrates a first perspective view of a tube securing device housing a tube in use on a patient in accordance with a first example embodiment of the present disclosure;

FIG. 23 illustrates an alternate view of a tube securing device housing a tube in use on a patient in accordance with a second example embodiment of the present disclosure;

FIG. 24 illustrates a tube comprising a first touch fastener in accordance with a third example embodiment of the present disclosure;

FIG. 25 illustrates a tube comprising a first and second section of a first touch fastener in accordance with a first example embodiment of the present disclosure;

FIG. 26 illustrates a tube securing device and a tube comprising first touch fastener material in accordance with a first example embodiment of the present disclosure;

FIG. 27 illustrates a first example embodiment comprising a structure to be housed within a channel of a tube securing device in accordance with an example embodiment of the present disclosure; and

FIG. 28 illustrates a second example embodiment comprising a structure to be housed within a channel of a tube securing device in accordance with an example embodiment of the present disclosure.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

The apparatus and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

### DETAILED DESCRIPTION

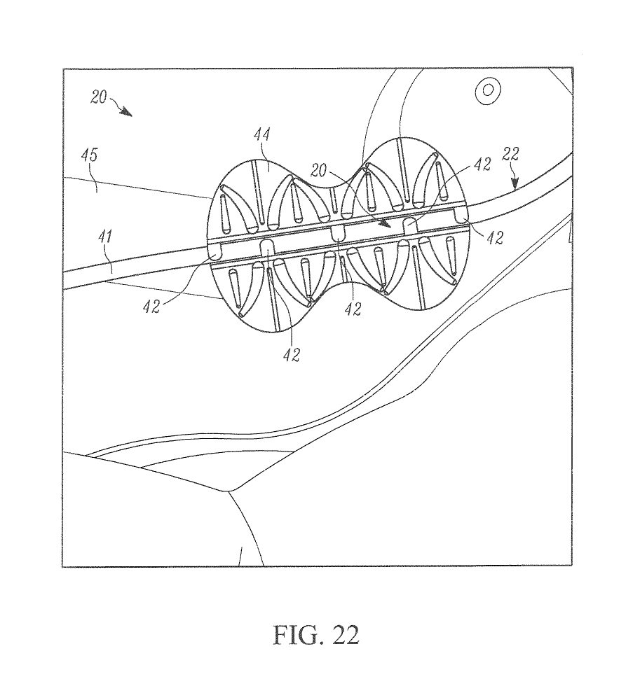

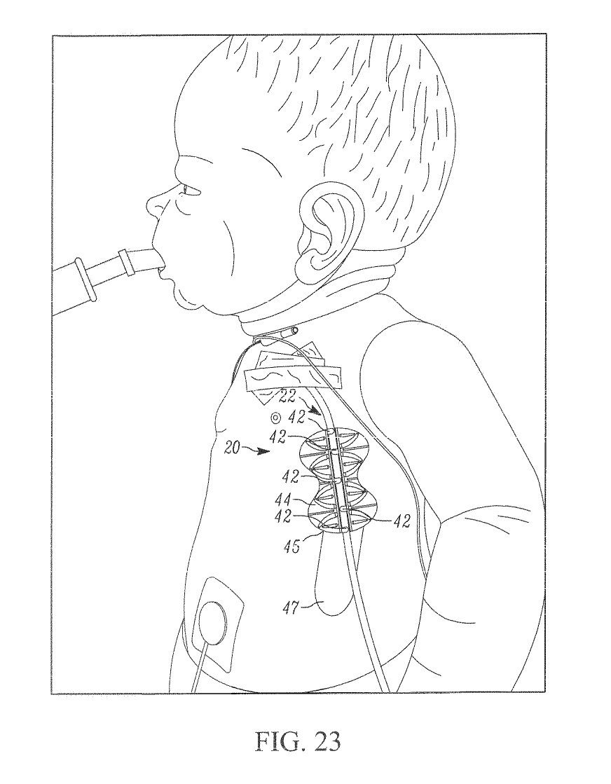

Referring now to the figures generally wherein like numbered features shown therein refer to like elements having similar characteristics and operational properties throughout unless otherwise noted. The present disclosure relates to a tube securing device 20. The tube securing device 20 can be used to anchor tubing 41 (e.g., chest tubing, facial tubing, tubing, such as catheters, central venous catheters, spinal catheters, ascetic tap catheters, peritoneal dialysis catheters, and the like, as illustrated in FIGS. 22-23 at a desired location on a patient's body before, during, and after a medical procedure.

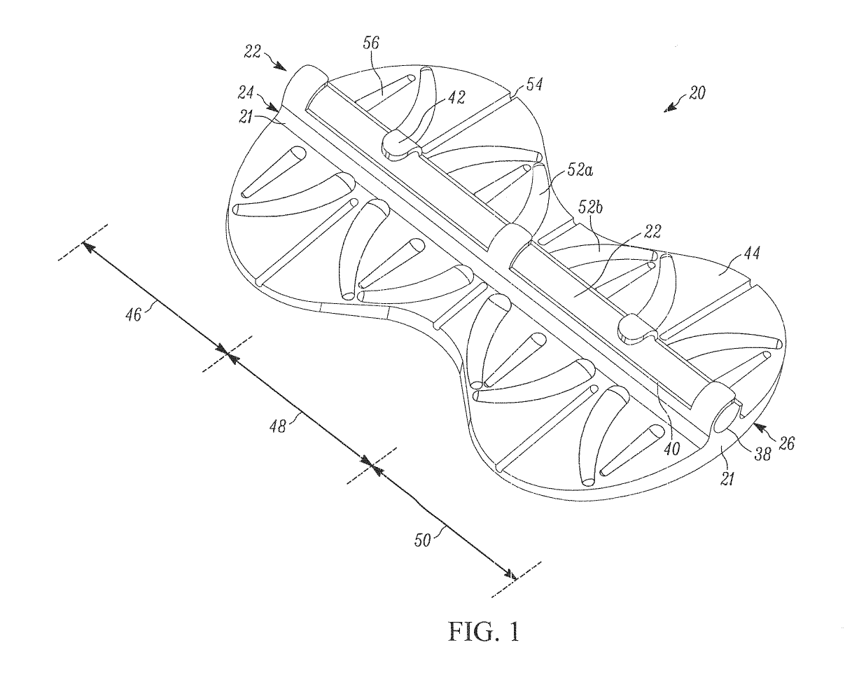

Referring to FIGS. 1-4 and FIGS. 22-23, in one example embodiment, the tube securing device 20 includes a tube receiving channel 22. The tube securing channel 22 is part of a body 21. In one example embodiment, the body 21 is formed from a polymeric material such as polypropylene, polyethylene, plastic and the like. The tube receiving channel 22 extends between a first end 24 and a second end 26 of the tube securing device 20 along a longitudinal axis 28. The longitudinal axis 28 divides the tube securing device 20 into a left side 30 and a right side 32. The terms “first end” 24, “second end” 26, “left side” 30, and “right side” 32 are used only to provide clarity to the description of the tube securing device 20 and do not in any way acid limitations to the tube securing device. Similarly, as used below, the terms “top side” 34 and “bottom side” 36 are also only used to provide clarity to the description of the tube securing device 20.

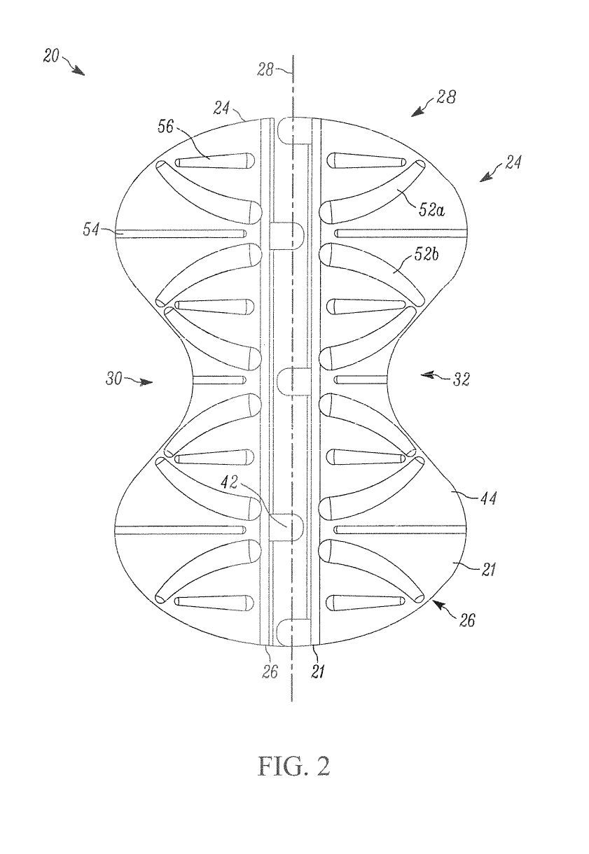

The tube receiving channel 22 includes a lower portion 38 and an upper portion 40. The lower portion 38 has a cross section that approximates a semi-circle having an inner diameter that is slightly larger than the tube 41. Illustrated in FIGS. 22-23, the tube securing device 20 is intended to anchor the tube 41 to a patient's body or skin. The lower portion 38 extends as an uninterrupted continuous surface along the longitudinal axis 28 between the first end 24 and the second end 26. The upper portion 40 also has a cross section that approximates a semi-circle, and has an inner diameter that is substantially equal to the inner diameter of the lower portion 38. As can be seen in FIGS. 1 and 2, the upper portion 40 is segmented into individual clasp 42 that are spaced apart from one another along the longitudinal axis 28 between the first end 24 and the second end 26 of the tube securing device 20.

In the illustrated example embodiment of FIG. 1, five clasps 42 are provided in an alternating arrangement on the left side 30 and the right side 32 of the tube securing device 20, and are configured to cover approximately 75% of the top half of a tube 41 retained in the tube receiving channel 22. However, in other example embodiments, a fewer or greater amount of clasps 42 are provided having any desired arrangement. For example, in another example embodiment, six clasps 42 are arranged such that three clasps are provided on the left side 30 in succession, followed by three clasps on the right side 32 in succession.

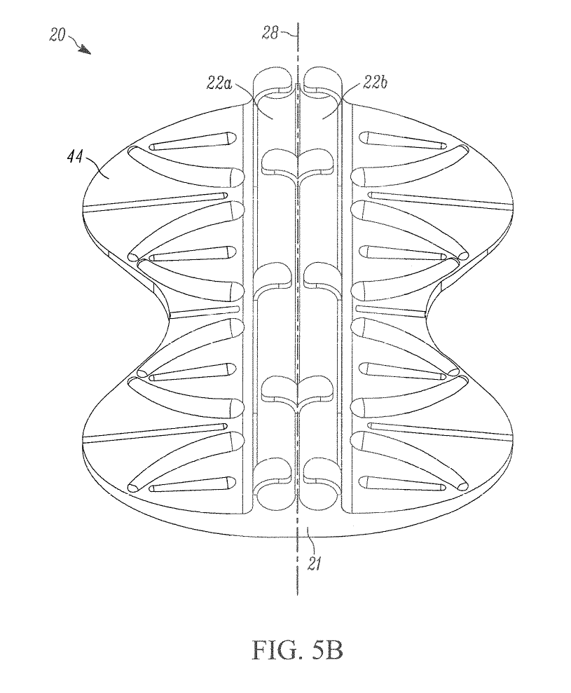

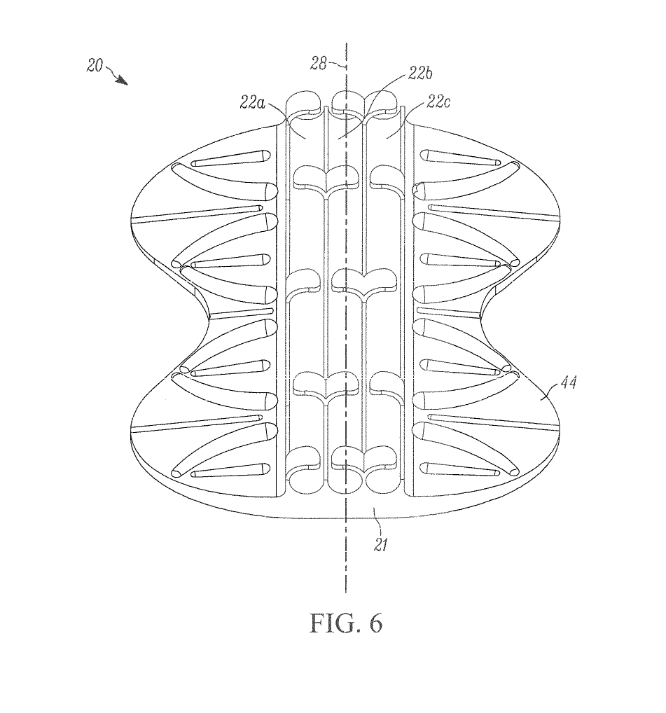

Each clasp 42 is formed by a flexible, resilient member, which facilitates easy insertion of the tube 41 into the tube receiving channel 22 while also ensuring that the tube 41 is securely retained by the tube securing device 20. In the example embodiment illustrated in FIGS. 1-4 and 5A the tube securing device includes a single tube receiving channel 22 that is aligned with the longitudinal axis 28. However, in other example embodiments, the tube securing device 20 includes two tube receiving channels 22a, 22b equally arranged on opposite sides of the longitudinal axis 28, as illustrated in FIG. 5A, or three tube receiving channels 22a, 22b, 22c as illustrated in FIG. 6, with one of the channels being aligned with the longitudinal axis 28 and the remaining two channels being equally arranged on opposite sides of the longitudinal axis 28. It is understood that in a further alternative example embodiment, the tube securing device 20 includes more than three tube receiving channels.

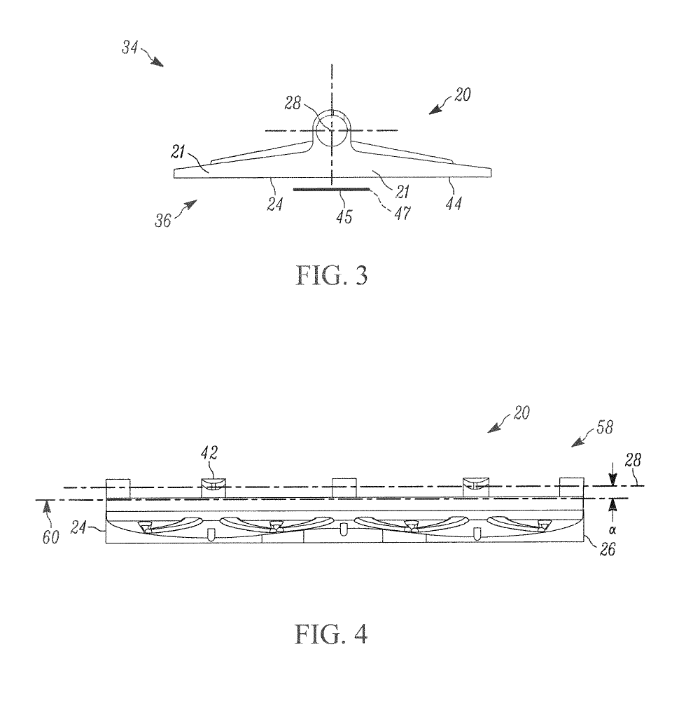

The body 21 is secured to a footprint portion 44 that is provided at on a bottom side of the body. In one example embodiment, the footprint portion 44 is formed of a polymeric material such as polypropylene, polyethylene, plastic and the like. When viewed from the perspective shown in FIG. 2, the footprint portion 44 has a shape that approximates an hourglass. In one example embodiment, a non-woven fabric (not shown) is attached to the footprint portion 44 at the bottom side 36 of the tube securing device 20. In another example embodiment, an adhesive pad is attached to the footprint portion 44 at the bottom side 36 of the tube securing device 20. The hourglass shape of the footprint portion 44 substantially divides the tube securing device 20 along the longitudinal axis 28 into a first end portion 46, a central portion 48, and a second end portion 50. As can be seen in FIG. 3, the footprint portion 44 is substantially planar and extends away from the longitudinal axis 28 equally toward the left side 30 and the right side 32.

The footprint portion 44 includes a plurality of reinforcement arms 52, full length relief grooves 54, and part length relief grooves 56. As can be seen in FIG. 1, the reinforcement arms 52 extend outwardly away from the lower portion 38 of the tube receiving channel 22 and toward either the left side 30 or the right side 32 of the tube securing device 20. The reinforcement arms 52 include first side facing arms 52a and second side facing arms 52b. As can be seen in FIG. 2, the first side facing arms 52a are arranged at a 45° angle relative to the longitudinal axis 28 and are slightly curved so as to point substantially toward the first end 24 of the tube securing device 20. The second side facing arms 52b are also arranged at a 45° angle relative to the longitudinal axis 28, but are slightly curved so as to point substantially toward the second end 26 of the tube securing device 20. The reinforcement arms 52 facilitate the maintenance of the structure and shape of the footprint portion 44 during use on a patient.

The full length relief grooves 54 are recesses in the body 21 and footprint portion 44 that extend outwardly from the lower portion 38 of the tube receiving channel 22 and intersect the perimeter of the footprint portion 44. The full length relief grooves 54 are provided between adjacent first side facing arms 52a and second side facing arms 52b that point away from one another, and are arranged to extend substantially perpendicular to the longitudinal axis 28.

The part length relief grooves 56 are recesses in the body 21 and footprint portion 44 that extend outwardly from the lower portion 38 of the tube receiving channel 22. In comparison to the full length relief grooves 54, the part length relief grooves 54 terminate just short of intersecting the perimeter of the foot print portion 44. The part length relief grooves 56 are provided between adjacent first side facing arms 52a and second side facing arms 52b that point, toward one another. Additional part length relief grooves 56 are provided at the first end 24 and the second end 26 of the securing device 20. The part length relief grooves 56 are arranged to extend substantially perpendicular to the longitudinal axis 28. The part length relief grooves 56 and full length relief grooves 54 facilitate flexibility of the footprint portion 44 during use on a patient, such that the footprint portion bends and flexes with movement of the patient, and to adhere to uneven surfaces comprised on the surface of the patient.

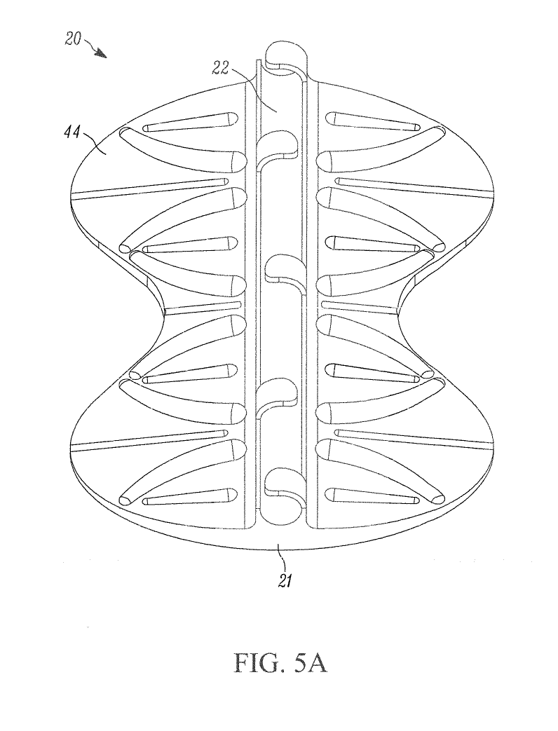

Referring to FIG. 4, a portion of the tube receiving channel 22 at the first end portion 46 of the tube securing device 20 is spaced away from the footprint portion 44 at a greater distance than a portion of the tube receiving channel 22 at the second end portion 50 of the tube securing device 20. This spacing arrangement results in a longitudinal axis 58 of the tube receiving channel 22 and a longitudinal axis 60 of the footprint portion 44 forming an acute angle α, which can promote fluid flow through the tube 41 retained by the tube securing device 20. However, it is understood that in another example embodiment, the longitudinal axis of the tube receiving channel and the longitudinal axis of the footprint portion are substantially parallel with one another.

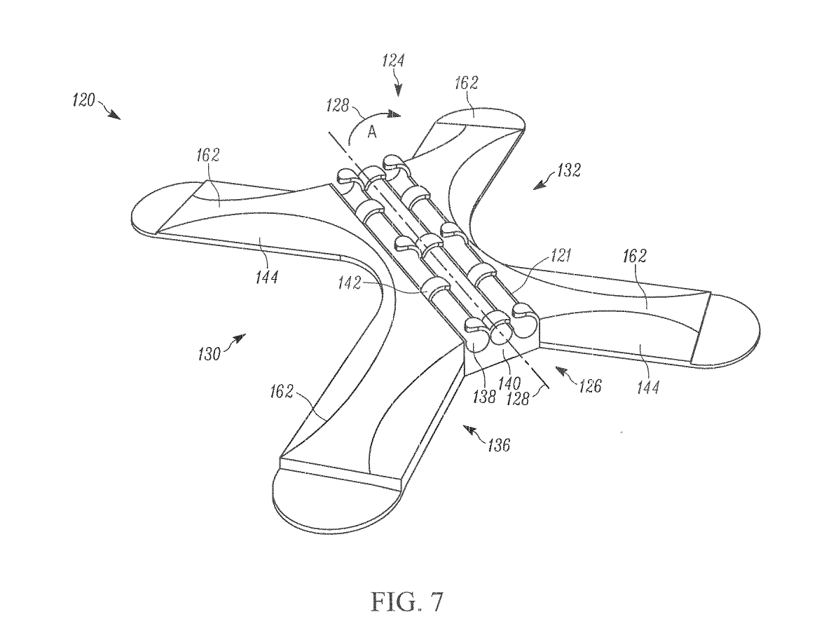

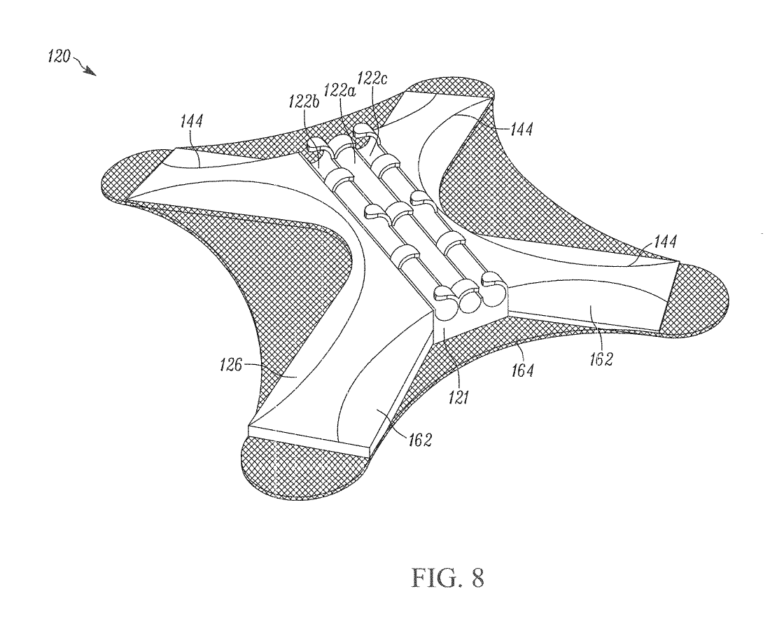

Referring now to FIGS. 7 and 8, another example embodiment of a tube securing 120 device is shown having a body 121. In one example embodiment, the body 121 is made from a polymeric material such as plastic having a durometer between 35-60 on a SHORE A scale. Features of the tube securing device 120 illustrated in FIGS. 7 and 8 that are similar to the features of the tube securing device 20 illustrated in FIGS. 1-4 and 22-23 will be identified by like numerals increased by a factor of one-hundred.

The tube securing device 120 includes three tube receiving channels 122a, 122b, 122c that extend between a first end 124 and a second end 126 of the tube securing device 120. Each of the three tube receiving channels 122 extends substantially parallel to a longitudinal axis 128 of the tube securing device 120. The longitudinal axis 128 divides the tube securing device 120 into a left side 130 and a right side 132. One of the tube receiving channels 122a is aligned with the longitudinal axis 128, and the remaining two channels 122b, 122c are equally arranged on opposite sides of the longitudinal axis 128. In alternative embodiments, the tube securing device includes fewer or greater tube receiving channels.

Each of the tube receiving channels 122 includes a lower portion 138 and an upper portion 140. The lower portion 138 extends as an uninterrupted continuous surface along the longitudinal axis 128 between the first end 124 and the second end 126. The upper portion 140 is segmented into spaced apart individual, flexible, resilient clasps 142 that extend along the longitudinal axis 128 between the first end 124 and the second end 126. Together, the upper portion 140 and the lower portion 138 have a substantially circular cross section having an inner diameter that is slightly larger than the tube 41 that the tube securing device 120 is intended to anchor in place.

The body 121 includes the tube receiving channels 122 that are secured to a footprint portion 144 that is provided at a bottom side of the body 121. The footprint portion 144 includes four arms 162 that are arranged so as to substantially form an “X” shape. Each of the arms 162 is part of the body 121 and are substantially resilient and flexible so as to be able to conform to a patient's body. In the illustrated embodiment the arms 162 are arranged such that, beginning at the longitudinal axis 128 at the first end 124 of the securing device 120 and moving rotationally clockwise in direction of arrow A, there is an arm 162 at approximately 45°, 135°, 225°, and 315°. However, in another example embodiment, the arms 162 are arranged at 90°, 180°, 270°, and 360° relative to the longitudinal axis at the first end 124 of the tube securing device 120. In yet another example embodiment, fewer or greater arras are provided and are arranged to form any desired shape with any desired spacing between the arms.

The securing device of FIG. 8 is substantially similar to the securing device of FIG. 7, with one notable difference being the presence of a mesh portion 164 that is attached to the body 121 at the bottom side 136 of the tube securing device 120. The mesh portion 164 serves to further facilitate the connection of the tube securing device 120 to a patient's skin.

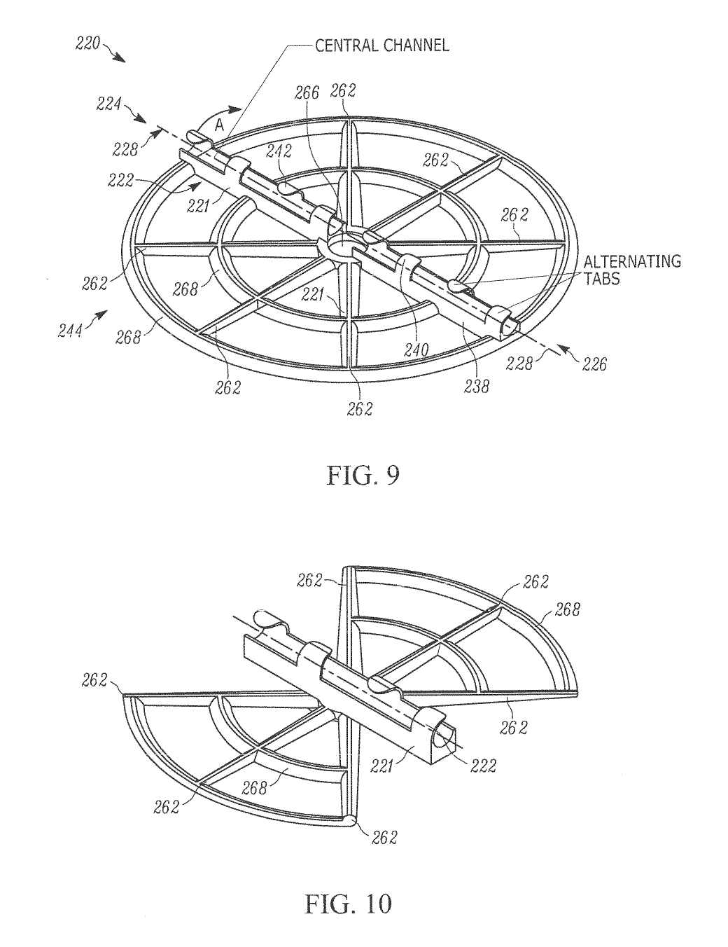

Referring to FIGS. 9 and 10, another example embodiment of a tube securing device 220 is shown and in one example embodiment formed from a polymeric material. Features of the tube securing device 220 illustrated in FIGS. 9 and 10 that are similar to the features of the tube securing device 20 illustrated in

FIGS. 1-4 and 22-23 will be identified by like numerals increased by a factor of two-hundred. The tube securing device 220 includes a body 221 comprising a tube receiving channel 222.

The tube receiving channel 222 extends between a first end 224 and a second end 226 of the tube securing device 220 along a longitudinal axis 228. The tube receiving channel 222 includes a lower portion 238 and an upper portion 240. The lower portion 238 extends as an uninterrupted continuous surface between the first end 224 and the second end 226 except for a central opening 266 provided midpoint between the first and second ends 224, 226. The upper portion 240 is segmented into spaced apart individual flexible, resilient clasps 242 that extend along the longitudinal axis 228 between the first end 224 and the second end 226. Together, the upper portion 240 and the lower portion 238 have a substantially circular cross section having an inner diameter that is slightly larger than the tube that the tube securing device 220 is intended to anchor in place.

The body 221 is secured to a footprint portion 244 that is provided at a bottom side of the body. The footprint portion 244 includes six arms 262 that extend radially outward from the central opening 266. Each of the arms 262 is substantially resilient and flexible so as to be able to conform to a patient's body or facial region,

In the illustrated embodiment, the arms 262 are arranged such that, beginning at the longitudinal axis 228 at the first end 224 of the tube securing device 220 and moving rotationally clockwise in the direction of arrow A, there is an arm at approximately 45°, 90°, 225°, 270°, and 315°. However, other configurations are possible. For example, in another embodiment, the tube securing device includes greater than six arms that are spaced at regular or irregular intervals relative to the longitudinal axis at the first end of the tube securing device. Reinforcing hoops 268 extend between adjacent arms 262. The reinforcing hoops 268 are concentric with the central opening 266. In the illustrated embodiment, two reinforcing hoops 268 are provided. However, in other example embodiments, fewer or greater reinforcing hoops are provided.

The securing device 220 of FIG. 10 is substantially similar to the securing device of FIG. 9, with one notable difference being that the central opening 266 is omitted. Another difference is that portions of the reinforcing hoops 268 are omitted. Specifically, the portions of the reinforcing hoops 268 that extend directly from the arms 262 to the tube receiving channel 222 are omitted.

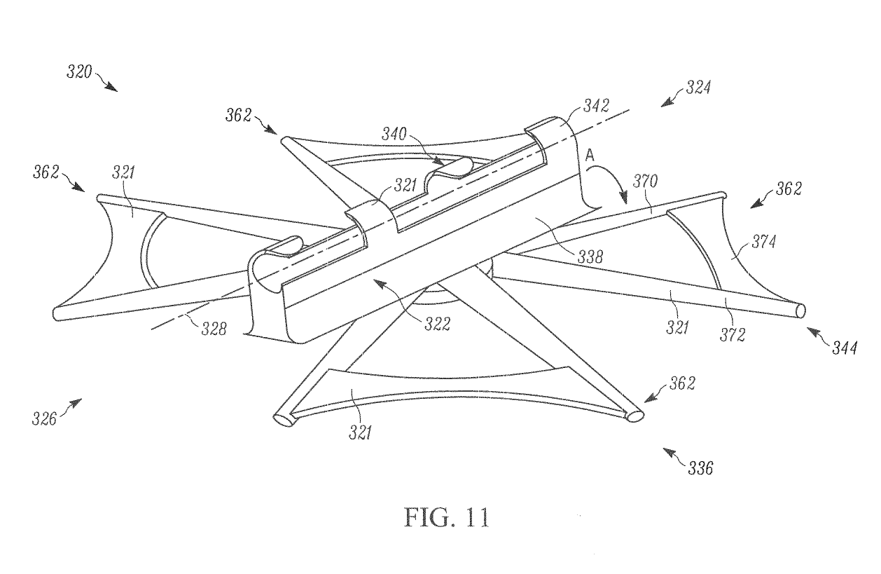

Referring now to FIG. 11, another example embodiment of a tube securing device 320 is shown. Features of the tube securing device 320 illustrated in FIG. 11 that are similar to the features of the tube securing device 20 illustrated in FIGS. 1-4 and 22-23 will be identified by like numerals increased by a factor of three-hundred.

The tube securing device 320 includes a body 321 having a tube receiving channel 322 that extends between a first end 324 and a second end 326 of the tube securing device 320 along a longitudinal axis 328. The tube receiving channel 322 includes a lower portion 338 and an upper portion 340. The lower portion 338 extends as an uninterrupted continuous surface along the longitudinal axis 328 between the first end 324 and the second end 326. The upper portion 340 is segmented into spaced apart individual flexible, resilient clasps 342 that extend along the longitudinal axis 328 between the first end 324 and the second end 326. Together, the upper portion 340 and the lower portion 338 have a substantially circular cross section having an inner diameter that is slightly larger than the tube that the tube securing device 320 is intended to anchor in place.

The body 321 is secured to a footprint portion 344 that is provided at a bottom side of the body. The footprint portion 344 includes four arms 362 that are arranged so as to substantially form an “X” shape. Each of the arms 362 is substantially resilient and flexible so as to be able to conform to a patient's body. In the illustrated embodiment, the arms 362 are arranged such that, beginning at the longitudinal axis 328 at the first end 324 of the tube securing device 320 and moving rotationally clockwise in a direction indicated by arrow A, there is an arm at approximately 45°, 135°, 225°, and 315°. However, other configurations are possible. For example, in another example embodiment, the arms are arranged at 90°, 180°, 270°, and 360° relative to the longitudinal axis 328 at the first end 324 of the tube securing device 320. Additionally, in another example embodiment, more than four arms are provided. Each arm 362 includes a first member 370 and a second member 372 with a bridge member 374 extending therebetween.

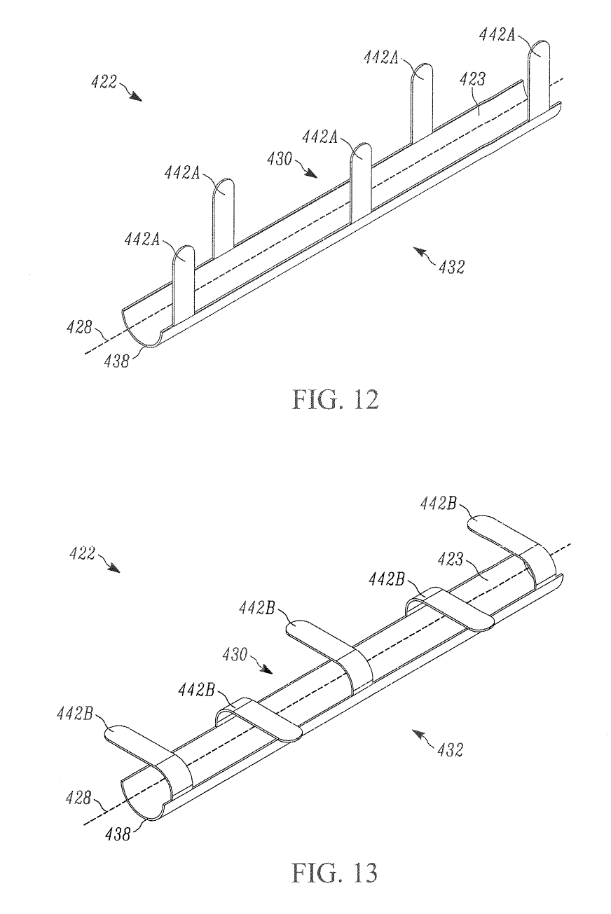

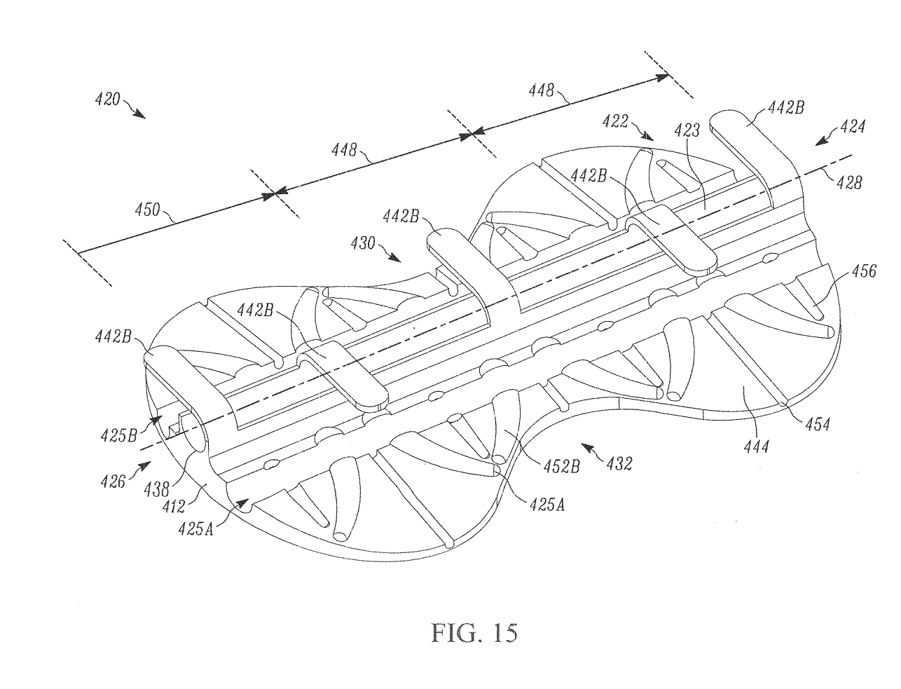

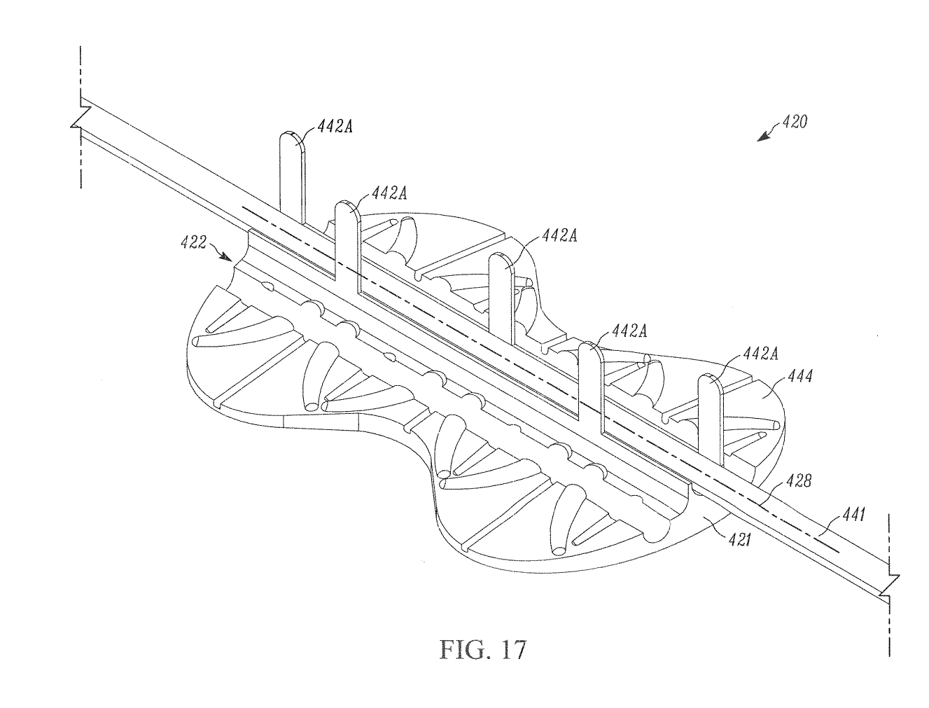

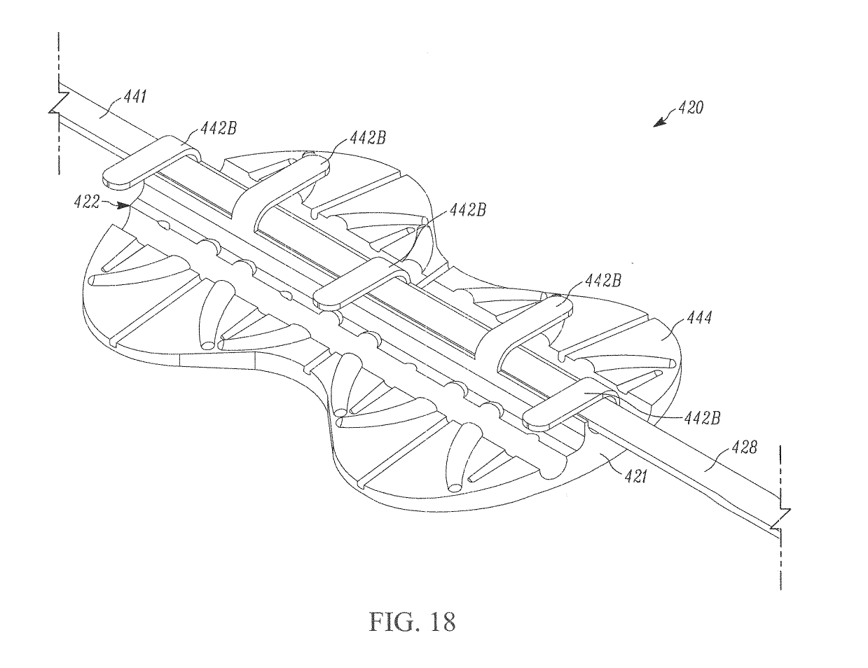

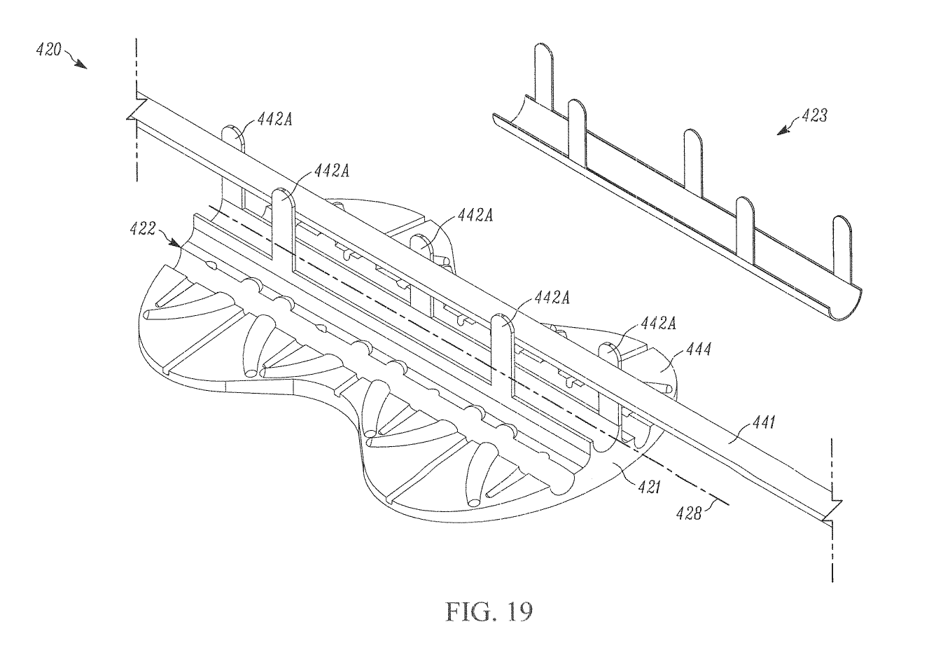

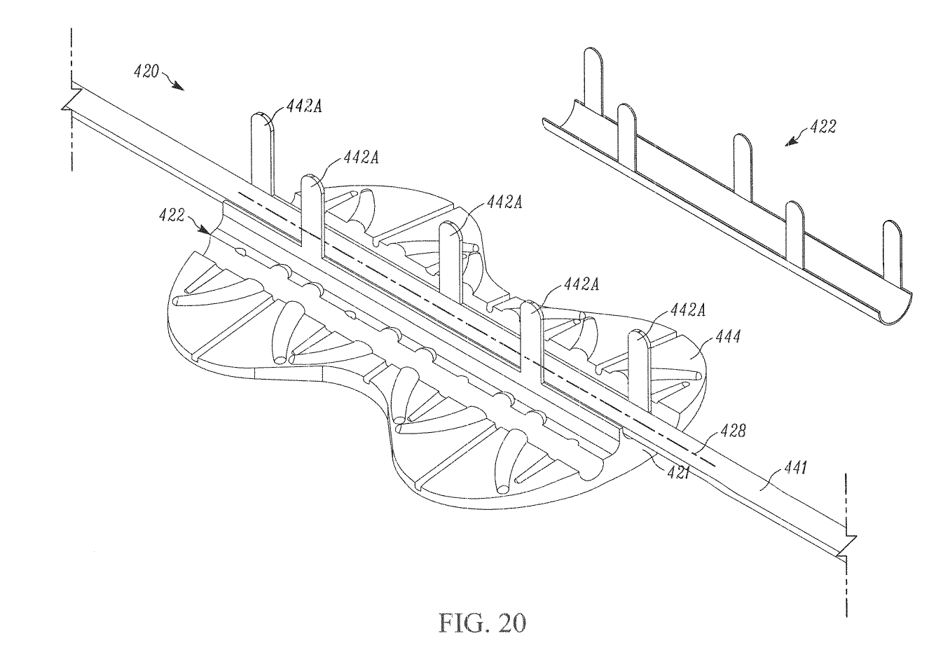

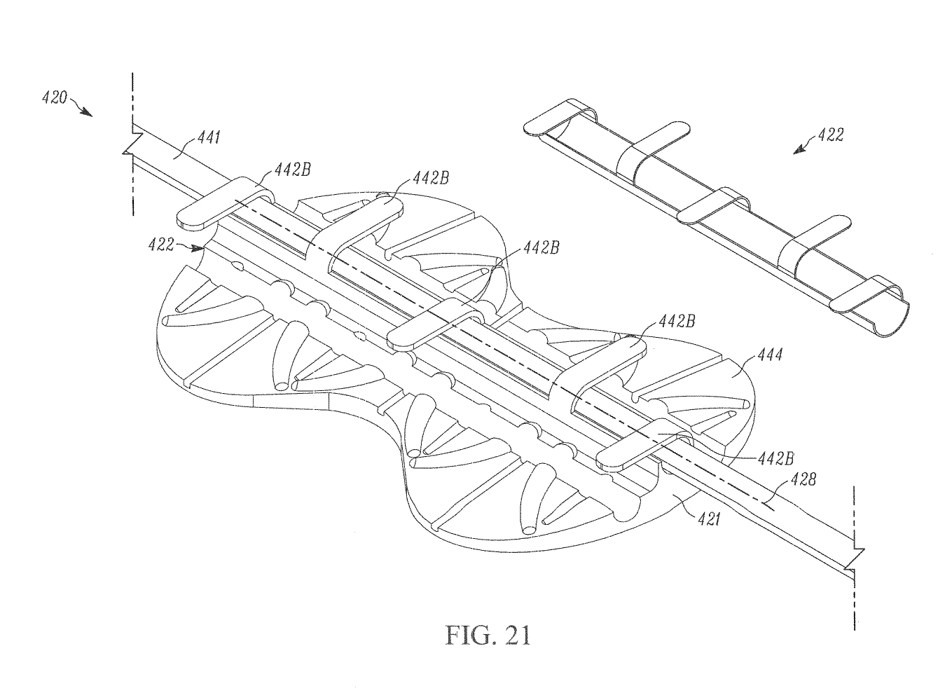

Referring now to FIGS. 12-21, another example embodiment of a tube securing device 420 is shown. Features of the tube securing device 420 illustrated in FIGS. 12-21 that are similar to the features of the securing device illustrated in FIGS. 1-4 and 22-23 will be identified by like numerals increased by a factor of four-hundred.

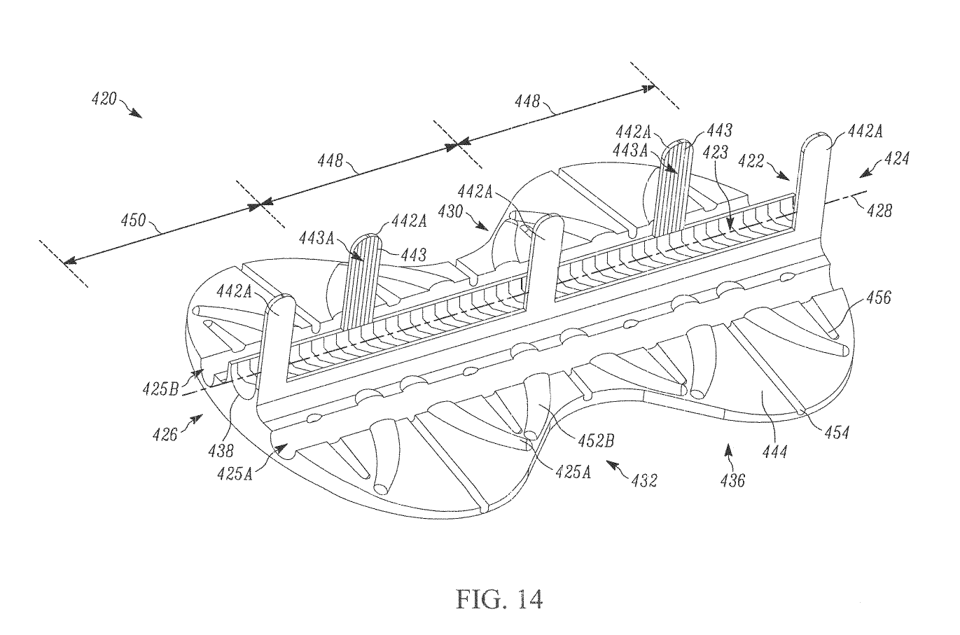

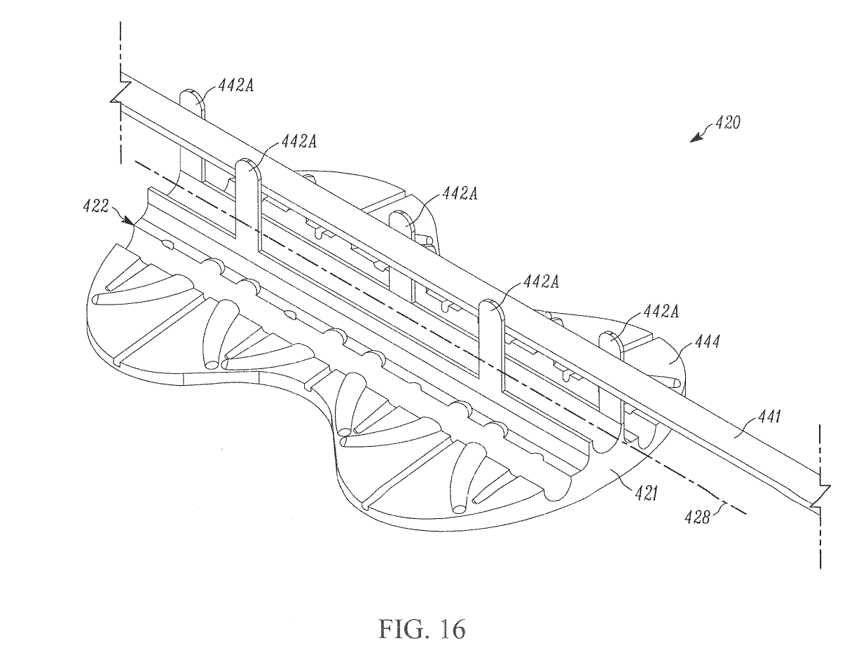

The securing device 420 of FIGS. 12-21 is substantially similar to the securing device of FIGS. 1-4 and 22-23, with a notable difference being that a body 421 comprises a tube receiving channel 422 comprising one or more bendable clasps 442. In the example embodiment, five bendable clasps 442 are provided in an alternating arrangement on a left side 430 and a right side 432 of the tube securing device 420. However, in other example embodiments, a fewer or greater number of bendable clasps 442 are provided having any desired arrangement. In the illustrated embodiment of FIG. 12, the bendable clasps 442A are in an open position, such as for receiving a tube 441, as illustrated in FIGS. 16-18, in the tube receiving channel 422 between the bendable clasps 442. In the illustrated embodiment of FIG. 13, the bendable clasps 442B are in a closed position, such as for retaining the tube 441 in the tube receiving channel 422.

In the illustrated example embodiment of FIG. 14, the body 421 is secured to a footprint portion 444 that is provided on a bottom side 436 of the body, there the footprint portion is substantially similar to the footprint portion 44 illustrated in FIGS. 1-4, 22-23. In another example embodiment the body 421 is connected to a footprint portion having a different configuration, such as the footprint portions 144, 244, 344, as illustrated in FIGS. 7-8, 9-10, and 11, respectively. In an example embodiment, the tube receiving channel 422 extends between a first end 424 and a second end 426 of the tube securing device 420 along a longitudinal axis 428.

As illustrated in FIGS. 16-18, the bendable clasps 442 bend to transition between the open and the closed position to allow for improved securing of the tube 441 in the tube receiving channel 422. In one example embodiment, the bendable clasps 442 comprise a pliant metal. In another example embodiment, the bendable clasps 442 comprises a silicon anchor coupled to the body 421 that has been co-molded around a ductile piece of steel or stainless steel. In an example embodiment, an inner surface 443, as illustrated in FIG. 14, of the bendable clasps 442 comprises inner ridges 443A transverse or perpendicular to the longitudinal axis 428. The inner ridges 443A limit movement of the tube 441. In the illustrated example embodiment of FIG. 14, the tube receiving channel 422 comprises inner channel ridges 423 along a lower portion 38 of the channel receiving tube 422, where the lower portion 38 extends along the longitudinal axis 28 between the first end 424 and the second end 426 of the tube receiving channel 422. The inner channel ridges 423 A are transverse or perpendicular to the longitudinal axis 428.

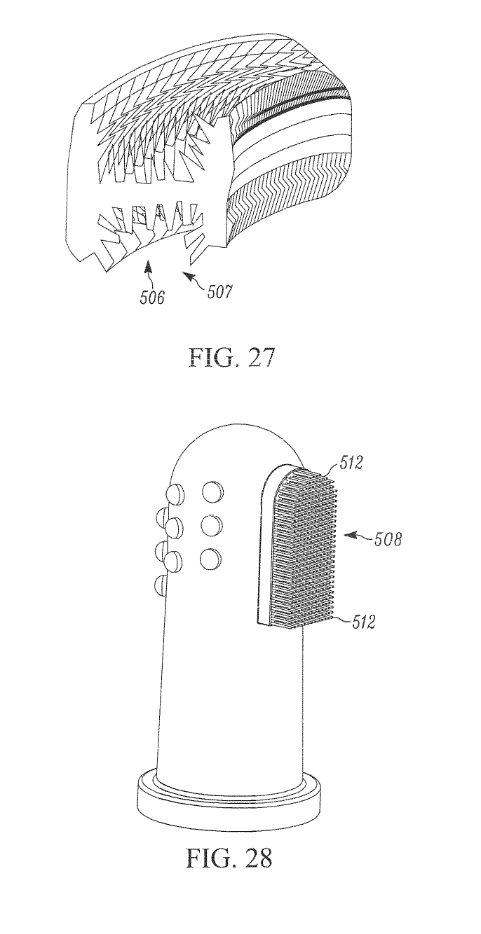

In one example embodiment, the inner channel ridges 423A comprise a series of first extended bodies 506 or second extended bodies 508 as illustrated FIGS. 27 or 28, respectively. The extended bodies can extend unidirectionally as illustrated in FIG. 28, or toward a common point, as illustrated in FIG. 27. The extended bodies 506, 508 are made from an interlocking material such as rubber, silicon, or plastic. The extended bodies 506, 508 form a more secure grip on the tube 441 to prevent the tube from shifting or moving within the channel 422. In another example embodiment, the extended bodies 506, 508 form the more secure grip on the tube 441 with interlocking fingers 507 comprising the inner channel ridges 423A, illustrated in FIG. 27, that engage extending fingers 512 coupled to the tube, illustrated in FIG. 28, to prevent the tube from shifting or moving within the channel 422. One of ordinary skill in the art would appreciate that the extending fingers 512 could comprise the inner channel ridges 423A and the interlocking fingers 507 could be coupled to the tube 441, or some variation thereof.

In another example embodiment, the footprint portion 444 comprises one or more additional tube receiving channels having a plurality of bendable clasps 442 (not shown). In one example embodiment, a second tube receiving channel, of the one or more additional tube receiving channels, is formed in a first groove 425 A of the footprint portion 444, and/or a third tube receiving channel, of the one or more additional clasp assemblies, is formed in a second groove 425B of the footprint portion 444. The first groove 425A and the second groove 425B are equally arranged on opposite sides of the longitudinal axis 428. In one example embodiment, the tube securing device 420 can house three or more tubes, in the tube receiving channel 222 and the one or more additional tube receiving channels. In one example embodiment, the bendable clasps 442 of the tube receiving channel 422 may be staggered relative to second bendable clasps (not shown) of the second tube receiving channel to prevent the bendable clasps 442 and the second bendable clasps from overlapping and/or interfering with each other.

As best seen in FIGS. 16-18, the tube 441 is inserted in the tube receiving channel 422 between the bendable clasps 442A in the open position. Once the tube 441 is housed in the tube receiving channel 422 between the bendable clasps 442A, the bendable clasps 442 are closed to comprise bendable clasps 442B in the closed position. The bendable clasps 442B secure the tube 441 in the tube receiving channel 422.

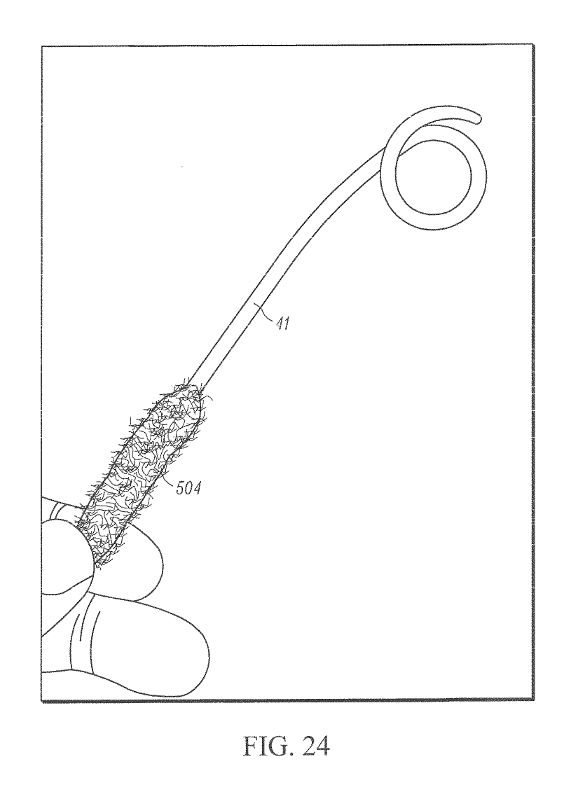

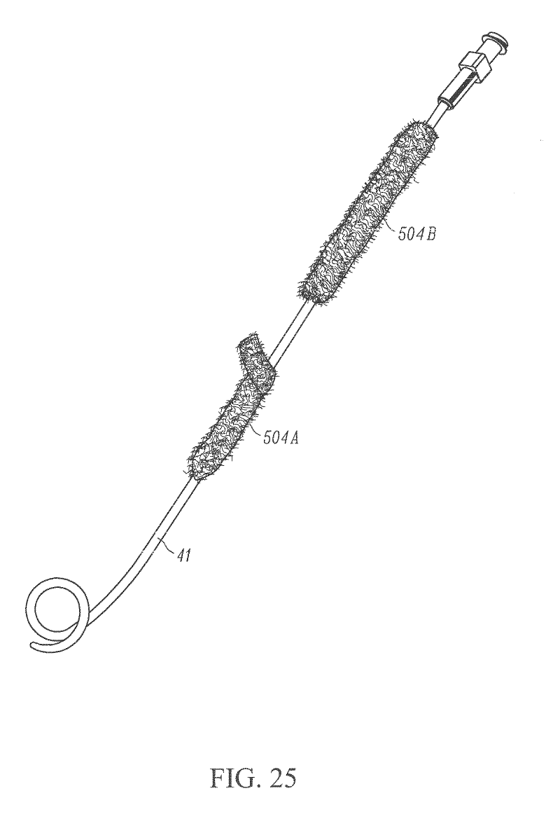

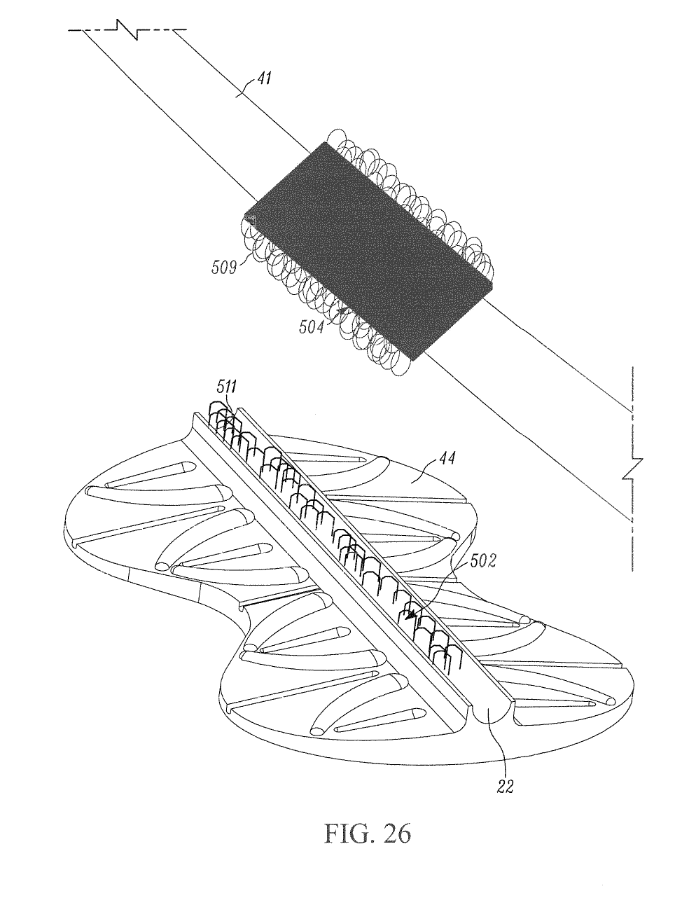

In the illustrated example embodiments of FIGS. 24-26, the tube 41 comprises at least one first touch fastener portion 504 of a first touch fastener material. One such first touch fastener is Velcro® manufactured by Velcro Industries B.V., having receiving and projecting forms (e.g., male and female) as would be appreciated by one of ordinary skill in the art. In another example embodiment, the at least one first touch fastener portion 504 material comprises a loop configuration 509 and a channel first touch fastener 502, located in the channel 22, comprises a hook configuration 511. In the illustrated example embodiment, the hook configuration 511 will interact and link with the loop configuration 509 to couple the tube 41 to the channel 22. In the illustrated example embodiment of FIG. 25, the tube 41 comprises a first first touch fastener portion 504A and a second first touch fastener portion 504B, to couple to channel first touch fasteners 502 in multiple channels 22 of multiple tube securing devices 20.

In each of the above embodiments, the tube securing device is manufactured as a single piece out of a polymeric, such as material having a durometer rating of SHORE A 35-60 hardness. However, in another example embodiment, the tube securing device 20-420 is manufactured out of several separate pieces that are subsequently assembled. Additionally, in another example embodiment, the tube securing device 20-420 is manufactured out of any suitable material that is flexible enough to allow the tube securing device to bend to the contours of a patient's body, yet stiff enough to resist inadvertent removal of a tube from the tube securing device.

Referring back to FIGS. 1-4 and 22-23, use of the tube securing device 20 will now be discussed. It should be noted that the use of the tube securing device is substantially the same for each of the above-described embodiments 120, 220, 320, and 420. Use of the device can be dependent on the age of the patient, with one method of use being appropriate for patients between 24 weeks-35 weeks old, and another method of use being appropriate for patients older than 35 weeks old. The different methods of use is due to the fact that patients younger than 35 weeks old typically do not have adequate skin development that would allow for proper attachment of the tube securing device of the present disclosure (or any other device for that matter) to the skin via an adhesive strip.

For patients younger than 35 weeks old or burn victims, or patients with similar weak skin tissue, a securing device 20 having non-woven fabric attached to the footprint portion 44 is utilized. Use of the securing device for the younger than 35 weeks old application begins by preparing an area of the patient's skin where the securing device 20 will be located with Marathon® Liquid skin protectant or other appropriate adhesive. The skin protectant is then allowed to dry. Then, the tube securing device 20 is placed on the patient such that the non-woven fabric is engaged with the dried skin protectant. Next, the interface between the non-woven fabric and the dried skin protectant is treated with additional liquid skin protectant. The additional liquid skin protectant impregnates the non-woven fabric and fuses the tube securing device 20 to the dried skin protectant, thereby attaching the tube securing device to the patient. The tube 41 can then be inserted into the tube receiving channel 22 as desired. When utilizing this method, removal of the tube securing device 20 can be achieved by way of a suitable solvent.

For patients older than 35 weeks old, a tube securing device 20 having an adhesive pad 45 attached to the footprint portion 44 is utilized, as illustrated in FIGS. 22-23. Use of the tube seeming device 20 for patients older than 35 weeks old begins with a clinician removing a protective film that is provided to cover the adhesive pad. Then, the tube securing device 20 is pressed against the patient such that the adhesive pad 45 is pressed against the patient's skin, thereby attaching the tube securing device 20 to the patient. To facilitate easy removal of the tube securing device 20, the adhesive pad 45 is of the type utilized in 3M Command™ Products. Removing the tube securing device 20 requires the clinician to firmly hold the tube securing device 20 against the patient's body while simultaneously pulling a provided tab 47 of the adhesive strip in a direction substantially parallel to the surface of the patient's skin. This action causes the adhesive pad 45 to release from the patient It is understood that, in another example embodiment any other suitable adhesive pad 45 is used.

In yet another example embodiment, the securing device 20 includes the body 21, the footprint portion 44, and the adhesive pad 45 coupled to a tab 47. The adhesive pad 45 includes an adhesive on the side attached to the footprint portion and on the opposite side of the pad that contacts the patient's body or skin. The tab 47 is coupled to the adhesive pad 45 in such a way that upon pulling on the tab in a direction parallel to the footprint portion, the shear stress to the adhesive has a lower yield point than the patient's skin or tissue, leaving the tissue or skin of the patient uncompromised on the patient side and the adhesive's low yield strength on the footprint side gives to break away from the footprint on the other side of the pad, hence releasing the securing device 20 from the patient.

In the foregoing specification, specific embodiments have been described. However, one of ordinary skill in the art appreciates that various modifications and changes can be made without departing from the scope of the disclosure as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The disclosure is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the terra is defined to be within 10%, in another embodiment within 5%, in another embodiment within 1% and in another embodiment within 0.5%. The term “coupled” as used herein is defined as connected or in contact, although not necessarily directly and not necessarily mechanically. A device or structure that is “configured” in a certain way is configured in at least that way, but may also be configured in ways that are not listed.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

What is claimed is:

1. A device tor securing medical tubing to a patient, comprising:
a body having a tube receiving channel extending along a longitudinal axis between a first end and a second end, the tube receiving channel including a lower portion and an upper portion, the upper portion having a plurality of flexible clasps that are spaced apart front one another along the longitudinal axis;
a footprint portion secured to the body;
a plurality of reinforcement arms provided on the footprint portion, each of the plurality of reinforcement arms being curved so as to point toward one of the first end and the second end; and
at least one of a non-woven fabric and an adhesive pad secured to the footprint portion.

2. The device of claim 1 wherein, the body comprises a second tube receiving channel extending between the first end and the second end, the tube receiving channel including a lower portion and an upper portion, the upper portion having a plurality of flexible clasps that are spaced apart from one another.

3. The device of claim 1 wherein the footprint portion comprises a substantially hour-glass shaped perimeter.

4. The device of claim 1 wherein the plurality of flexible clasps are provided in an alternating arrangement on alternating sides of the tube receiving channel.

5. The device of claim 1 wherein each of the plurality of flexible clasps comprise a bendable portion, said bendable portion having at least a 85° bending angle.

6. The device of claim 4 wherein each of the plurality of flexible clasps comprise a c-shape, wherein each of said flexible clasps extend partially over the tube receiving channel.

7. The device of claim 6 wherein each of the plurality of flexible clasps comprises a clasp end, opposite an attached end of the c-shape. wherein the attached end is coupled to a first side of the tube receiving channel, said clasp end a first distance from a second side of the tube receiving channel the second side opposite the first side, the first distance configured to be less than a diameter of a tube to be attached.

8. The device of claim 1 wherein the tube receiving channel comprises a first touch material within said channel configured to interact with a first touch material on a tube when in use.

9. The device of claim 1 wherein the tube receiving channel comprises a textured surface that comprises flexible protrusions configured to grip a tube.

10. The device of claim 1 wherein the tube receiving channel comprises a series of protrusions that are transverse to the longitudinal axis configured to grip a tube.

11. The device of claim 1 wherein a plurality of relief grooves is provided on the foot print portion, a single groove of the plurality of relieve grooves being provided between adjacent reinforcement arms of the plurality of reinforcement arms.

12. The device of claim 1 wherein the tube receiving channel comprises flexible protrusions converging toward a point that is centrally located relative to said tube receiving channel, said flexible protrusions configured to grip a tube.

13. A device for securing medical tubing to a patient, comprising:
a body having a tube receiving channel extending along a longitudinal axis between a first end and a second end, the tube receiving channel including a lower portion and an upper portion, the upper portion having a plurality of flexible clasps that are spaced apart from one another along the longitudinal axis;
a footprint portion secured to the body, the footprint portion including a plurality of arms extending radially outward from the body between the first end and the second end along the longitudinal axis; and
at least one of a non-woven fabric and an adhesive pad secured to the footprint portion.

14. The device of claim 13, wherein the adhesive pad comprises at least one tab, the at least one tab coupled to an adhesive portion of the adhesive pad, wherein responsive to a force being applied to said at least one tab in a direction parallel to the longitudinal axis the adhesive portion is removed from a patient and the footprint without compromising a skin of the patient.

15. The device of claim 14, wherein the footprint portion further comprises a plurality of reinforcement hoops, the reinforcement hoops being concentric about the midpoint and interconnecting adjacent arms of die plurality of arms.

16. The device of claim 13, wherein the plurality of arms extend outwardly relative to the longitudinal axis at an angle between 30° to 90°.

17. The device of claim 13 wherein, the body comprises one or more additional tube receiving channels extending between the first end and the second end, the one or more additional tube receiving channels including a lower portion and an upper portion, the upper portion having a plurality of flexible clasps that are spaced apart from one another.

18. The device of claim 13 wherein the tube receiving channel comprises a first touch material within said channel.

19. The device of claim 13 wherein the rube receiving channel comprises a textured surface that comprises flexible protrusions configured to grip a tube.

20. A method of securing medical tubing to a patient comprising the steps of:
providing a device having a tube receiving channel that includes a lower portion and an upper portion, the upper portion having a plurality of flexible clasps that are spaced apart from one another along a longitudinal axis, a footprint portion secured to the tube receiving channel, and a non-woven fabric attached to the footprint portion;
applying liquid skin protectant to a desired area of the patient's skin and allowing the liquid protectant to dry to form a solid protectant;
placing the securing device on the patient such that the non-woven fabric engages the solid protectant;
applying additional liquid protectant to the interlace between the non-woven fabric and the solid protectant so as to impregnate the non-woven fabric with the additional liquid protectant and allowing the additional liquid protectant to dry; and
inserting a medical tube into the tube receiving channel.
